(19) United States
(12) Patent Application Publication
(10) Pub. No.: US 2016/0310523 A1
(43) Pub. Date: Oct. 27, 2016

(54) USE OF OXYGEN-CONTAINING GAS SOURCE

(71) Applicant: China Medical University, Taichung City (TW)

(72) Inventor: Wei-Ta LAI (Ted LAI), Taichung City (TW)

(21) Appl. No.: 14/846,841

(22) Filed: Sep. 7, 2015

(30) Foreign Application Priority Data
Apr. 23, 2015 (TW) 104113060

(51) Int. Cl. A61K 33/00

(52) U.S. Cl. CPC A61K 33/00

(57) ABSTRACT

A method of increasing blood-brain barrier permeability of a subject to a molecule is disclosed. The method includes causing the subject to inhale a gas source, and the gas source being effective for increasing blood-brain barrier permeability to the molecule, wherein the gas source includes at least 20 weight percent oxygen.

### RELATED APPLICATIONS

This application claims priority to Taiwan Application Serial Number 104113060, filed Apr. 23, 2015, which is herein incorporated by reference.

### BACKGROUND

1. Technical Field

The present disclosure relates to the use of an oxygen-containing gas source. More particularly, the present disclosure relates to the use of the oxygen-containing gas source for increasing blood-brain barrier permeability of a subject to a molecule.

2. Description of Related Art

A blood-brain barrier is a tremendously tight-knit layer of endothelial cells that coats of capillaries and blood vessels in a brain. The nearly impermeable junctions between blood-brain barrier cells are formed by an interdigitation of about 20 different types of proteins. Foreign molecules need to enter the blood-brain barrier cell through membrane-embedded protein transporters or by slipping directly through its waxy outer membrane. Once inside, foreign compounds need to avoid a high concentration of metabolic enzymes and a variety of promiscuous protein pumps primed to eliminate foreign substances. Having avoided these obstacles, foreign molecules need to then pass through an inner membrane of the blood-brain barrier cell to finally reach the brain.

The capillaries that supply a blood to tissues of the brain constitute the blood-brain barrier. The endothelial cells which form the brain capillaries are different from those found in other tissues in the body. Brain capillary endothelial cells are joined together by tight intercellular junctions which form a continuous wall against the passive diffusion of molecules from the blood to the brain and other parts of a central nervous system. These cells are also different in that they have few pinocytic vesicles which in other tissues allow somewhat unselective transport across the capillary wall. Also lacking are continuous gaps or channels running between the cells which would allow unrestricted passage.

The blood-brain barrier functions to ensure that an environment of the brain is constantly controlled. The levels of various substances in the blood, such as hormones, amino acids, and ions, undergo frequent small fluctuations which can be brought about by activities such as eating and exercise. If the brain was not protected by the blood-brain barrier from these variations in serum composition, the result could be uncontrolled neural activity.

Although it is believed that the blood-brain barrier serves a protective function under normal conditions by protecting the brain from exposure to potentially hazards, in brain diseases, the blood-brain barrier may thwart therapeutic efforts by hindering the entry of neurotherapeutic compounds into the brain. For example, although many bacterial and fungal infections may be readily treated where the site of the infection is outside the central nervous system, such infections in the central nervous system are often very dangerous and very difficult to treat due to the inability to deliver an effective doses of drugs to the site of the infection. Similarly, the action of the blood-brain barrier makes a treatment of cancers of the brain more difficult than the treatment of the cancers located outside the central nervous system. Even where it may be possible to deliver the effective dose of drug into the central nervous system by administering very large amounts of drug outside of the central nervous system, the drug levels outside the central nervous system (such as in the blood) are then often so high as to reach toxic levels deleterious to kidneys, a liver, and other vital organs.

The typical methods for increasing the blood-brain barrier permeability include an intra-arterial injection of hyperosmolar solutions (via common carotid artery) and an ultrasound stimulation that are able to improve the problem of the compounds delivery into the central nervous system. However, the intra-carotid injection is an invasive treatment that has potential risks, and the ultrasound stimulation requires expensive equipment that is not widely available. In addition, both the intra-carotid injection and the ultrasound stimulation mediated blood-brain barrier disruption suffer poor reversible and uncontrollable outcomes, so that it takes a long time to recover impermeability of the blood-brain barrier after the blood-brain barrier disruption mediated by these methods.

### SUMMARY

According to one aspect of the present disclosure, a method of increasing blood-brain barrier permeability of a subject to a molecule includes causing the subject to inhale a gas source, and the gas source being effective for increasing blood-brain barrier permeability to the molecule, wherein the gas source includes at least 20 weight percent oxygen.

### BRIEF DESCRIPTION OF THE DRAWINGS

The present disclosure can be more fully understood by reading the following detailed description of the embodiment, with reference made to the accompanying drawings as follows:

FIG. 1A is a photograph showing a Evans blue dye (EBD) extravasation in rats stimulated by different stimulation frequencies;

FIG. 1B is a quantitative diagram showing the detection amount of EBD in a motor cortex of the rats;

FIG. 1C is a quantitative diagram showing the detection amount of EBD in a blood of the rats;

FIG. 2A is a schematic view showing test groups of a novel object recognition test;

FIG. 2B is a flaw schematic view of the novel object recognition test;

FIG. 2C is a quantitative diagram showing a number of the rats contact with an object in the novel object recognition test, wherein the rats are injected EBD on the test day;

FIG. 2D is a quantitative diagram showing the detection amount of EBD in different districts of a brain and the blood of the rats;

FIG. 2E is a quantitative diagram showing a number of the rats contact with the object in the novel object recognition test, wherein the rats are injected sodium fluorescein (NaF) on a test day;

FIG. 2F is a quantitative diagram showing the detection amount of NaF in different districts of the brain and the blood of the rats;

FIG. 3A is a quantitative diagram showing the detection amount of EBD in different districts of the brain and the blood of the rats in an elevated platform test;

FIG. 3B is a quantitative diagram showing the detection amount of EBD in different districts of the brain and the blood of the rats in a forced swimming test;

FIG. 3C is a quantitative diagram showing the detection amount of EBD in different districts of the brain and the blood of the rats, wherein the rats are injected EBD 30 minutes after behavioral activities;

FIG. 3D is a quantitative diagram showing the detection amount of NaF in different districts of the brain and the blood of the rats in the behavioral activities;

FIG. 4A illustrates the effect of drugs on a blood-brain barrier opening of the rats in the forced swimming test;

FIG. 4B is a quantitative diagram showing the detection amount of EBD in different districts of the brain and the blood of the rats in a hypercapnia test;

FIG. 4C is a quantitative diagram showing the detection amount of EBD in the motor cortex and the blood of the rats injected Na⁺/H⁺ exchanger (NHE);

FIG. 5A is a quantitative diagram showing the detection amount of EBD in different districts of the brain and the blood of the rats in a claustrophobia test;

FIG. 5B is a quantitative diagram showing the detection amount of EBD in different districts of the brain and the blood of the rats in an immobilization test;

FIG. 5C illustrates concentration changes of corticosterone in the rats in stress associated with behavior activity tests;

FIG. 5D illustrates O₂/CO₂ content in the rats in the stress associated with behavior activity tests;

FIG. 6A is a quantitative diagram showing the detection amount of EBD in different districts of the brain of the rats in the claustrophobia test;

FIG. 6B is a quantitative diagram showing the detection amount of EBD in the blood of the rats in the claustrophobia test;

FIG. 7A is a photograph showing the EBD extravasation in the rats breathed different gas sources;

FIG. 7B is a quantitative diagram showing the detection amount of EBD in different districts of the brain and the blood of the rats;

FIG. 8A is a photograph showing the EBD extravasation in the rats following different level of ventilation;

FIG. 8B is a quantitative diagram showing the detection amount of EBD in different districts of the brain and the blood of the rats in the hypoventilation;

FIG. 8C is a quantitative diagram showing the detection amount of EBD in different districts of the brain and the blood of the rats increased a ventilation rate;

FIG. 9A is a quantitative diagram showing the detection amount of EBD in different districts of the brain of the rats inhaled different gas sources;

FIG. 9B is a quantitative diagram showing the detection amount of EBD in the blood of the rats inhaled different gas sources;

FIG. 9C is a quantitative diagram showing the detection amount of EBD in different districts of the brain of the rats inhaled 20% carbogen for different exposure time;

FIG. 9D is a quantitative diagram showing the detection amount of EBD in the blood of the rats inhaled 20% carbogen for different exposure time;

FIG. 9E is a quantitative diagram showing the detection amount of EBD in a liver of the rats inhaled 20% carbon for different exposure time;

FIG. 9F is a photograph showing the EBD extravasation in the rats inhaled 20% carbogen in different exposure time;

FIG. 10A is a quantitative diagram showing the detection amount of EBD in different districts of the brain and the blood of the rats during recovery from 30 minutes of 20% carbogen inhalation;

FIG. 10B is a quantitative diagram showing the detection amount of EBD in different districts of the brain and the blood of the rats during recovery from 60 minutes of 20% carbogen inhalation;

FIG. 10C is a quantitative diagram showing the detection amount of EBD in different districts of the brain and the blood of the rats during recovery from 90 minutes of 20% carbogen inhalation;

FIG. 11A is a quantitative diagram showing body weight changes of the rats inhaled 20% carbogen;

FIG. 11B is a quantitative diagram showing a food intake of the rats inhaled 20% carbogen;

FIG. 11C is a quantitative diagram showing the result of a locomotor activity on Day 20, wherein the rats inhale 20% carbogen;

FIG. 11D is a quantitative diagram of the result of the novel object recognition test on Day 22, wherein the rats inhale 20% carbogen;

FIG. 12A is a flow schematic view of the novel subject recognition test, wherein the rats received the carbogen inhalation and a ZIP peptide injection on Day1;

FIG. 126 is a quantitative diagram of the result of the novel object recognition test on Day 1;

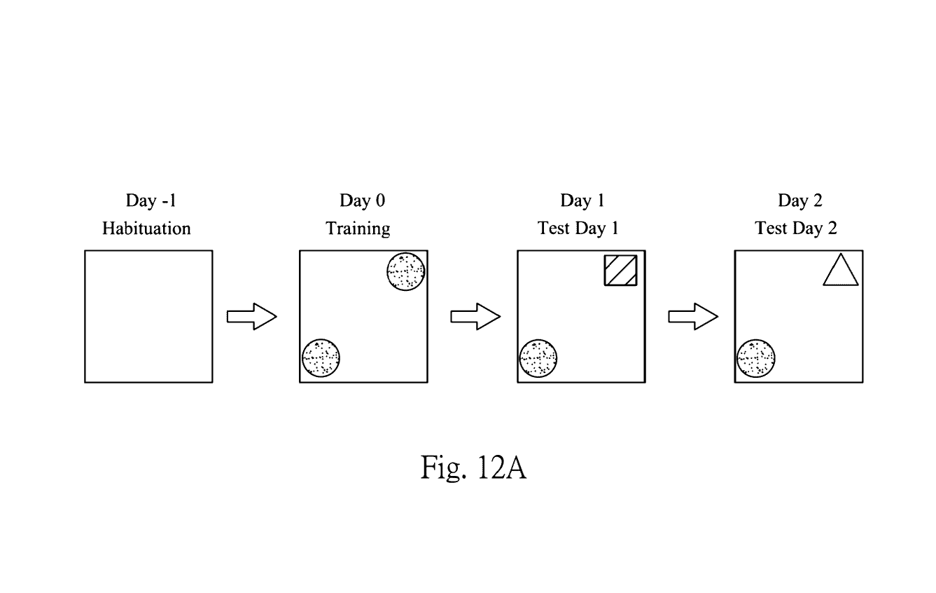

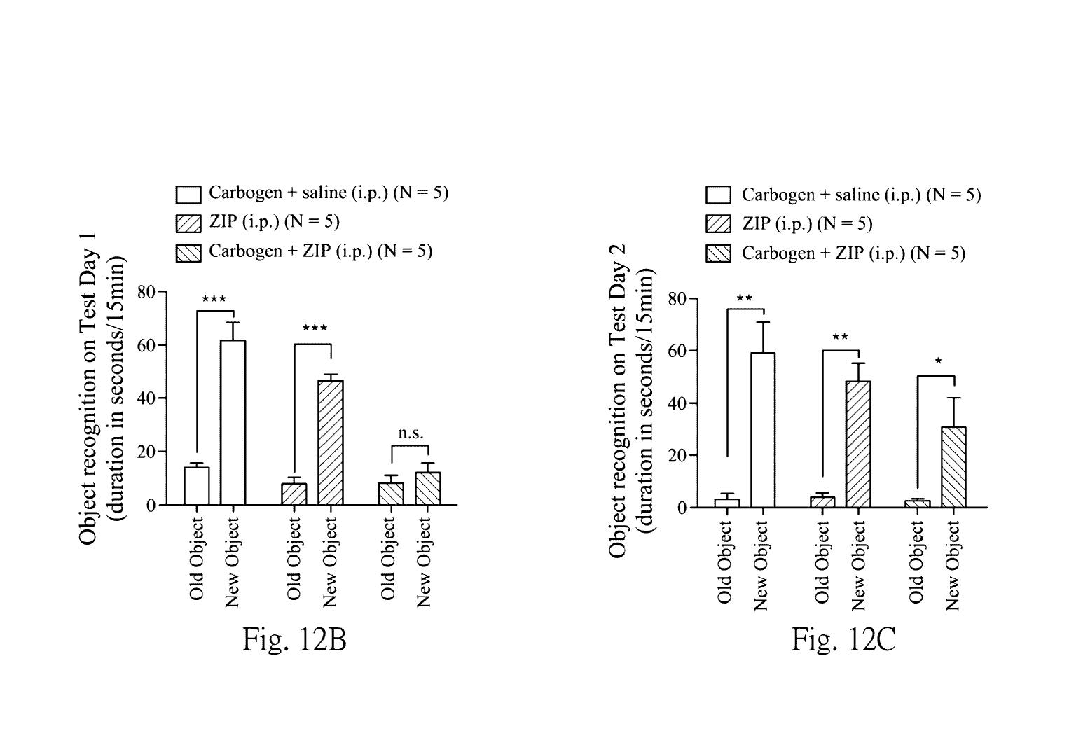

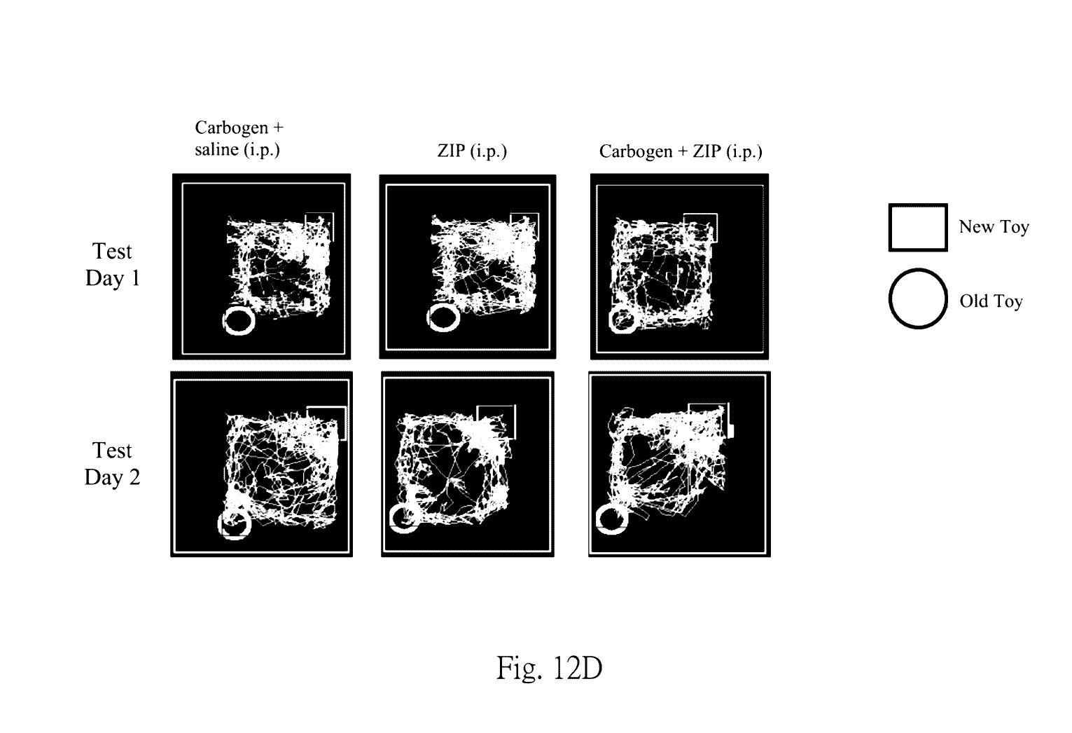

FIG. 12C is a quantitative diagram of the result of the novel object recognition test on Day 2; and

FIG. 120 is a schematic diagram of the result of the novel object recognition test.

### DETAILED DESCRIPTION

A novel use of a gas source which contains oxygen (O₂) is provided. According to earlier results of regardless in vivo animal experiment models, aforementioned gas source is capable of increasing a blood-brain barrier permeability of a subject to a molecule. Thus the gas source is a potentially effective therapeutic agent for increasing the blood-brain barrier permeability. The following are descriptions of the specific terms used in the specification:

The term “gas source” refers to a gas mixture included at least 20 weight percent O₂. The gas source can further include 1 weight percent carbon dioxide (CO₂), wherein a ratio of O₂ weight/volume percent (w/v %) to CO₂ (w/v %) ranges from 1:1 to 1:20. The weight/volume percent (w/v %) is defined as [mass of solute (g)/volume of solution (ml)]×100.

The term “blood-brain barrier disruption” or “blood-brain barrier opening” means the increase in the blood-brain barrier permeability, hence foreign molecules, which would be shut out of a blood-brain barrier, being able to enter a brain through the blood-brain barrier.

The term “carbogen” refers to the gas mixture consisted of O₂ and CO₂. The carbogen has been used in clinical treatment for psychiatric disorders. For instance, treatment regimens have included an inhalation of 40% carbogen (40% CO₂ with 60% O₂) for a period of 5 to 20 minutes, which is acutely effective against catatonia, or brief inhalation of 30% carbogen (30% CO₂ with 70% O₂) 3-6 times per week for a total 20-150 clinical visits, which has shown long-term efficacy in a treatment of several types of neurotic disorders.

Reference will now be made in detail to the present embodiments of the present disclosure, examples of which are illustrated in the accompanying drawings. Wherever possible, the same reference numbers are used in the drawings and the description to refer to the same or like parts.

### Examples

### I. Effect Factors of the Blood-Brain Barrier Disruption

Reasons affected the blood-brain barrier disruption are complex, and therefore this part of the examples first discuss that what individual physiological conditions are the effect factors of the blood-brain barrier disruption.

### (1). Pattern of Evoked Neuronal Activity Affects Blood-Brain Barrier Permeability

This example first evaluated whether the blood-brain barrier permeability can be affected by different patterns of evoked neuronal activity. Regulated neuronal activity can be elicited by repeated neuronal firing at a specific frequency. For instance, a high frequency stimulation (HFS) at 100 Hz for 1 second causes a long-term potentiation (LTP) of synaptic efficacy, which has been implicated for spatial learning, fear conditioning, and drug addiction. In addition, a low frequency stimulation (LFS) at 1-5 Hz for 5-30 minutes causes a long-term depression (LTD) of synaptic efficacy, which has been implicated for memory retrieval, reward craving, and cognitive processing. In marked comparison, a very low frequency stimulation (VLFS) at <0.02 Hz is akin to spontaneous neuronal activity that does not cause synaptic changes and is not associated with functional output of a brain.

To compare whether different patterns of neuronal activity confer distinct the blood-brain barrier permeability, in this example, test animals are stimulated by different stimulation frequencies, and is observed the effect of the stimulation on the blood-brain barrier disruption of the test animals. The test animals are male Sprague-Dawley (SD) rats, weighing 225-275 g, which are purchased from BioLASCO Co. Ltd. (Taipei, Taiwan). The rats are stimulated by LFS (120 stimuli per minute) or VLFS (1 stimulus per minute) and then examined the blood-brain barrier permeability to the Evans blue dye (EBD), EBD is water-soluble and has a very high affinity for serum albumin to form a 69 KDa protein tracer and thereby acts as an intravascular protein tracer during evoked extracellular stimulation by either. HFS is not examined in this example because of its short duration (typically 1 second), which would preclude sufficient EBD extravasation for accurate quantitation by spectroscopic methods.

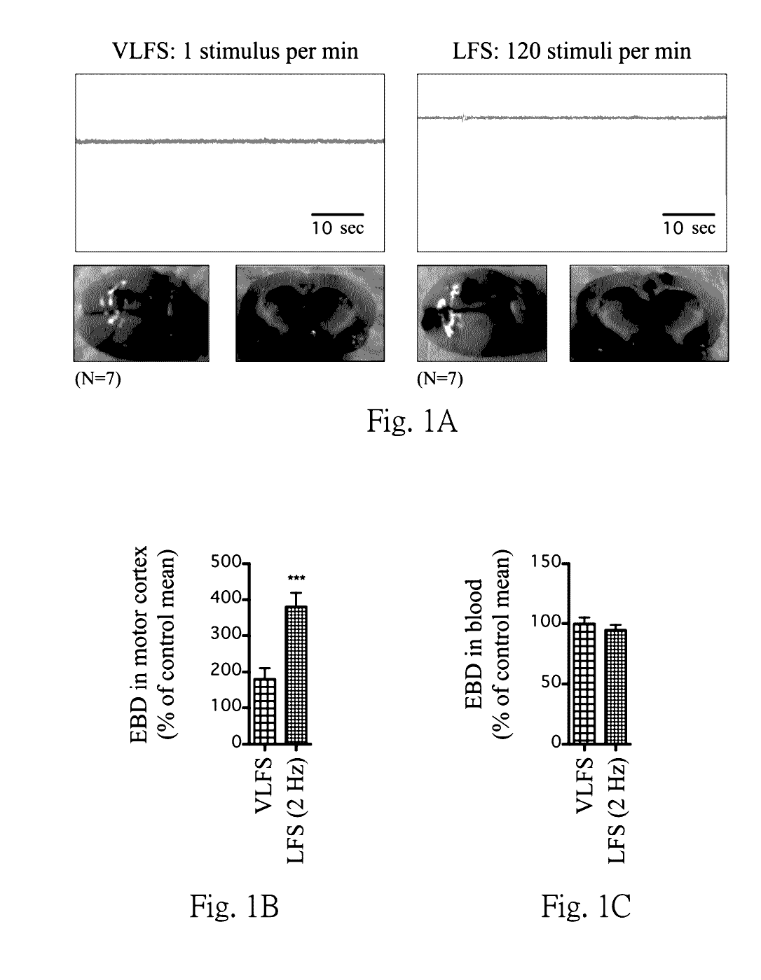

FIGS. 1A-1C show the effects of different patterns of neuronal activity on the blood-brain barrier permeability. FIG. 1A is a photograph showing the EBD extravasation in the rats stimulated by different stimulation frequencies. FIG. 1B is a quantitative diagram showing the detection amount of EBD in a motor cortex (MC) of the rats. FIG. 1C is a quantitative diagram showing the detection amount of EBD in a blood of the rats. Neuronal activity is evoked by a stimulus electrode placed in the motor cortex of urethane-anesthetized rats that received EBD injection, and is confirmed by extracellular recording with a recording electrode, wherein a current amplitude or a pulse width is no chage from VLFS to LFS. Stimulus evoked changes in the blood-brain barrier permeability are indicated by extravasation of the EBD into an ipsilateral in comparison to a contralateral hemisphere. These results demonstrated that increasing stimulus frequency per se increased the blood-brain barrier permeability in the stimulated hemisphere. These data suggest that different patterns of neuronal activity could confer distinct the blood-brain barrier permeability. However, the patterns of neuronal activity could induce different blood-brain barrier permeability is the type that contributes to LTD-related brain function (as with LFS) rather than the type that occurs spontaneously in the resting brain (as with VLFS).

### (2). Behavioral Activity in Conscious Rat Opens the Blood-Brain Barrier of Active Brain Regions

Given that local neuronal activity evoked by electrodes can be very different from integrated network activity that confers conscious behavior; this example next investigates whether behavioral activity can open the blood-brain barrier in brain regions thought to be involved in a metabolism. The rats that received either one of two tracers, EBD for tracing leakage of plasma protein and sodium fluorescein (NaF) for tracing the extravasation of small charged molecule, are subjected to 1 of 3 behavioral activities: a novel object recognition test, an elevated platform test, or a forced swim test.

### i. Novel Object Recognition Test

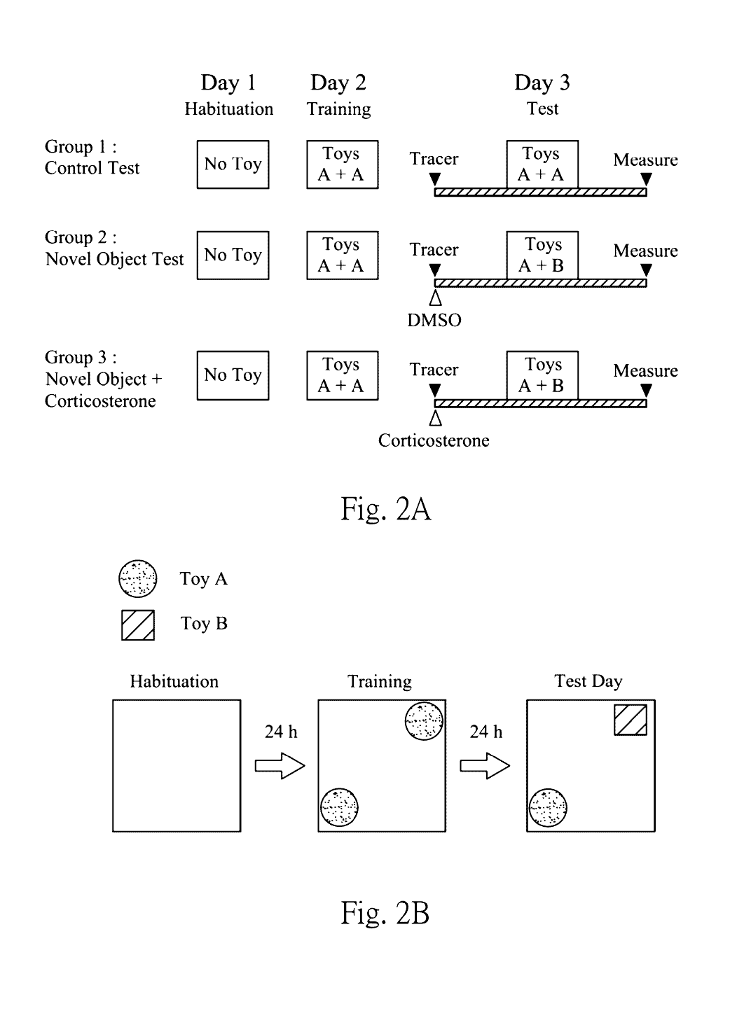

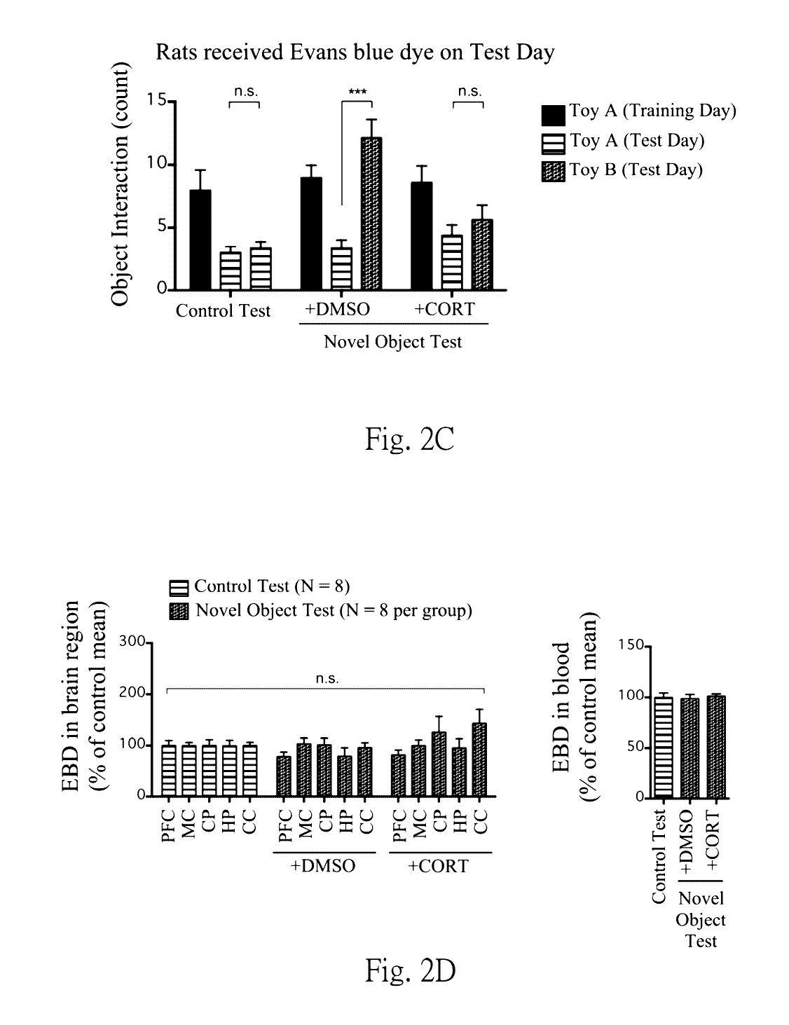

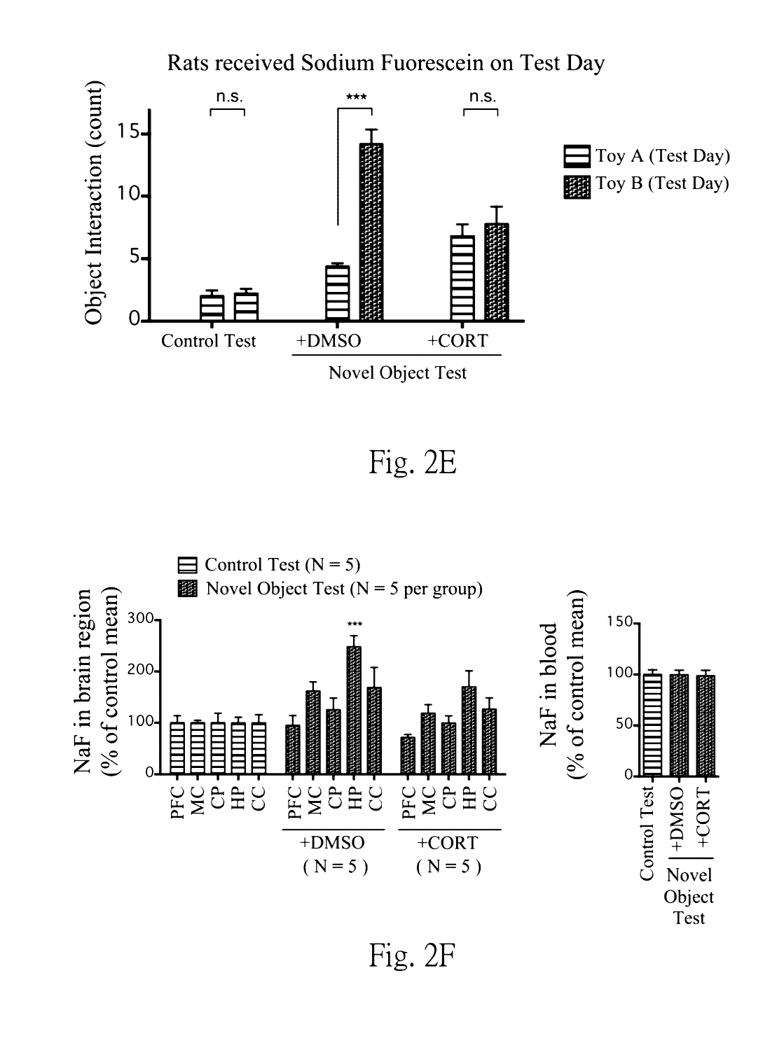

FIGS. 2A-2F show the results of the novel object recognition test. FIG. 2A is a schematic view showing test groups of a novel object recognition test. FIG. 2B is a flow schematic view of the novel object recognition test. FIG. 2C is a quantitative diagram showing a number of the rats contact with the object in the novel object recognition test, wherein the rats are injected EBD on the test day. FIG. 2D is a quantitative diagram showing the detection amount of EBD in different districts of the brain and the blood of the rats. FIG. 2E is a quantitative diagram showing a number of the rats contact with the object in the novel object recognition test, wherein the rats are injected NaF on the test day.

The novel object recognition test is widely used to test the memory retrieval function. In the first day of the novel object recognition test, the rats are placed in an open space, where is used for the novel object recognition test, to adapt 30 minutes. In the second day of the novel object recognition test, two identical objects (toys A, A) are placed in the open space and the rats are measured a touch time of two identical objects within 5 minutes. The rats, which touch any one object less than 5 seconds, are eliminated from the novel object recognition test to prevent an individual activity difference of the rats or a situation of unable recognition memory occurrence caused by lack of time. In the third day of the novel object recognition test, one of the two identical objects (toys A, A) is replaced with a novel object (toy B). The rats are trained to spend more time playing (recognizing) the new object (toy B) over the older object (toy A), and then the rats are measured the touch time of two different objects within 5 minutes. There are 3 groups in test groups. In a group 1, control group, two identical objects (toys A, A) are placed in the open space on the third day of the novel object recognition test. In a group 2 and a group 3, experiment groups, two different objects (toys A, B) are placed in the open space on the third day of the novel object recognition test, but the rats of the group 2 are injected dimethyl sulfoxide (DMSO) before the novel object recognition test and the rats of the group 3 are injected corticosterone before the novel object recognition test.

In FIG. 2D and FIG. 2F, the rats show no evidence for leakage of plasma protein (traced by EBD), but significant increase in the extravasation of small molecules (traced by NaF) into a hippocampus (HP) during the memory retrieval task. The hippocampus is a region of the brain associated with memory retrieval; hence the specific opening of the blood-brain barrier in hippocampus is consistent with the crucial role of this brain region for novel object recognition. When spatial memory retrieval is interrupted by injection of corticosterone, which has been shown to mimic stress-induced memory impairment, the blood-brain barrier opening is also attenuated.

### ii. Elevated Platform Test

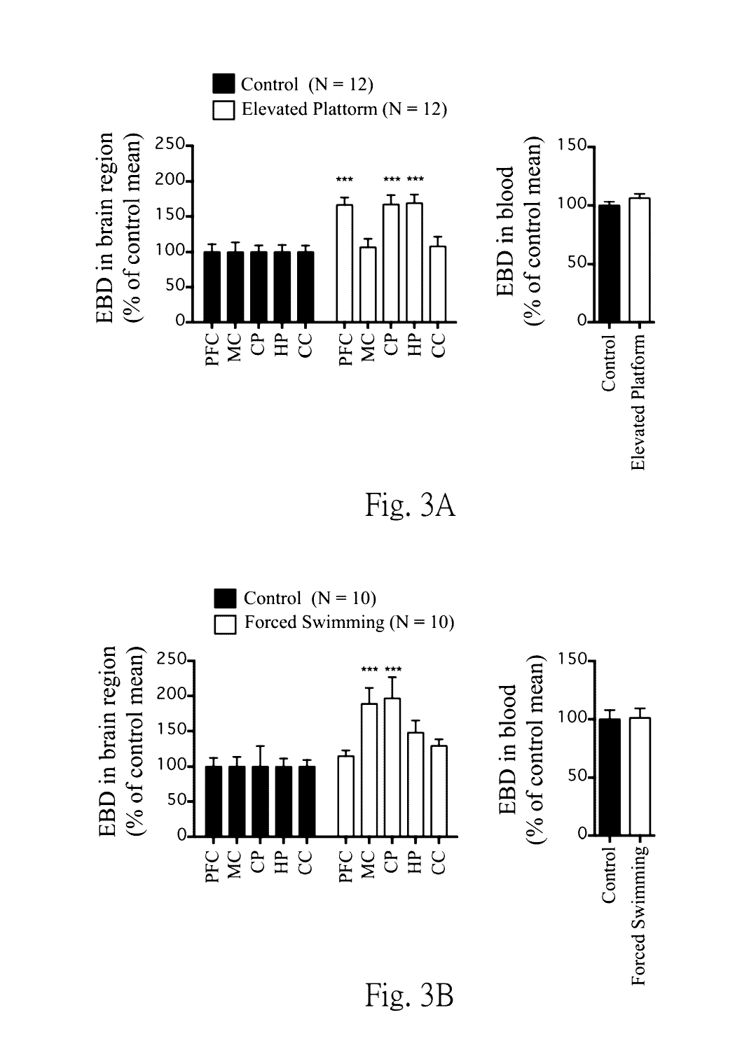

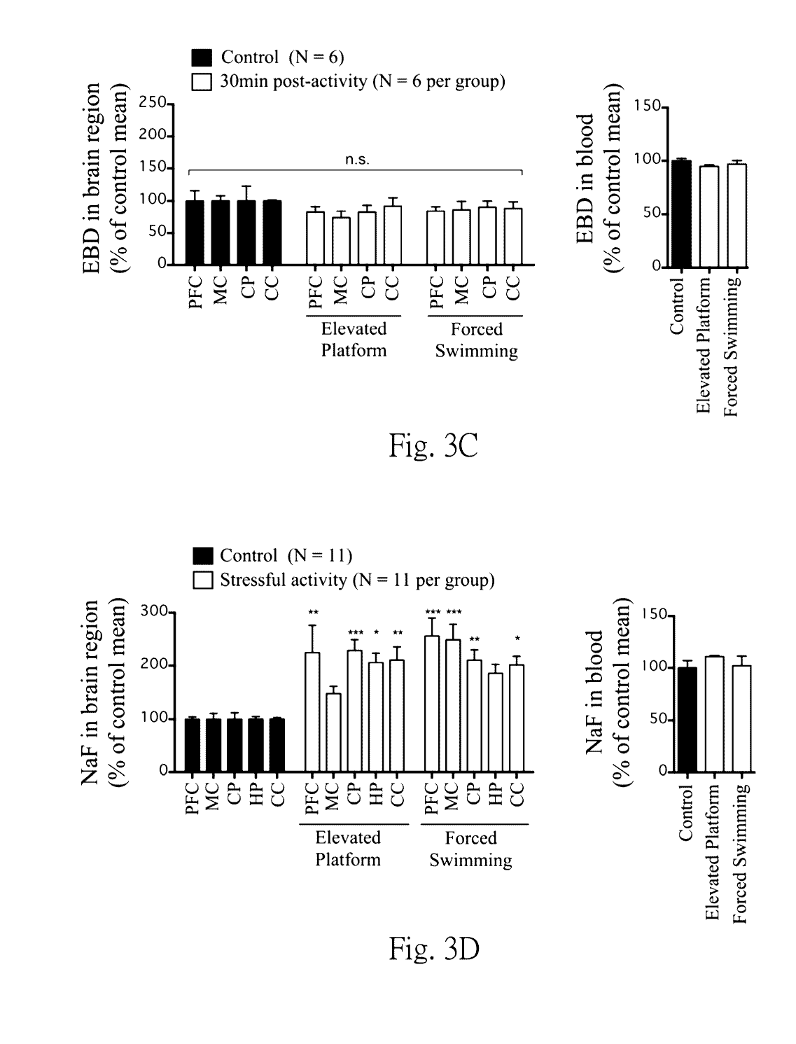

An elevated platform test is a modified elevated plus maze, in which the rats are forced to explore a spatial environment on a tall translucent platform. It will result in a state of anxiety in the rats. The rats are injected tracers before the elevated platform test. FIG. 3A is a quantitative diagram showing the detection amount of EBD in different districts of the brain and the blood of the rats in an elevated platform test. FIG. 3C is a quantitative diagram showing the detection amount of EBD in different districts of the brain and the blood of the rats, wherein the rats are injected EBD 30 minutes after behavioral activities. FIG. 3D is a quantitative diagram showing the detection amount of NaF in different districts of the brain and the blood of the rats in the behavioral activities. In the elevated platform test, the rats show significant increase in the blood-brain barrier permeability to both plasma protein (traced by EBD) and small molecules (traced by NaF), specifically in a prefrontal cortex (PSF), a caudate/putamen (CP), the hippocampus, and a cerebellum (CC), wherein the blood-brain barrier permeability in the cerebellum is increased to small molecules (NaF) only. The opening of the blood-brain barrier in the prefrontal cortex and the hippocampus is consistent with spatial learning, whereas the opening in the caudate/putamen and the cerebellum is consistent with a motor coordination to keep from failing off the platform. The results of this example are consistent with neuronal activity-dependency. In FIG. 3C, the opening of the blood-brain barrier appears to be temporally restricted, as no opening is observed if the tracer is injected 30 minutes after the test.

### iii. Forced Swim Test

In a forced swim test, the rats are forced to swim in an opaque tank with limited spatial information, wherein a diameter, a height and a depth of the opaque tank is 43 cm, 54 cm and 29 cm, respectively. FIG. 3B is a quantitative diagram showing the detection amount of EBD in different districts of the brain and the blood of the rats in the forced swimming test. FIG. 3C is a quantitative diagram showing the detection amount of EBD in different districts of the brain and the blood of the rats, wherein the rats are injected EBD 30 minutes after behavioral activities. FIG. 3D is a quantitative diagram showing the detection amount of NaF in different districts of the brain and the blood of the rats in the behavioral activities. In the forced swimming test, the rats show significant increase in the blood-brain barrier permeability to either tracers in the motor cortex and the caudate/putamen, regions known to be important for motor output, and also the prefrontal cortex and cerebellum (NaF only), The results of this test are consistent with dependence on ongoing neuronal activity. In FIG. 3C, no opening is observed when the tracer is injected 30 minutes after the test.

Taken together, these data demonstrates that the blood-brain barrier permeability is increased specifically in the brain regions required for each behavior. In addition, the lack of the blood-brain barrier opening 30 minutes following the behavioral activities reveal the transient nature of this phenomenon.

### (3). Functional Hyperemia and Blood-Brain Barrier Opening Shares Similar Mediator

Cellular metabolism results in local hypercapniaiacidosis, and this regional increase in CO₂/H⁺ is believed to the primary mediator of functional hyperemia to peripheral organs such as a heart. The brain also uses neuron-specific mechanisms, such as N-methy-D-aspartate receptor (NMDAR)-mediated release of nitric oxide, astrocyte-specific mechanisms, and pericyte-specific mechanisms to trigger functional cerebral hyperemia. Of these, this example is particularly interested in whether the NMDAR pathway contributes to the blood-brain barrier opening. The elevated platform test and the forced swim test are stressful, and stimulation of the RU486-sensitive receptor by these tests has been shown to enhance NMDAR signaling and thereby affect brain function. Thus, this could explain why it observes more severe the blood-brain barrier opening following the elevated platform and the forced swim tests, in comparison to the less stressful object recognition test.

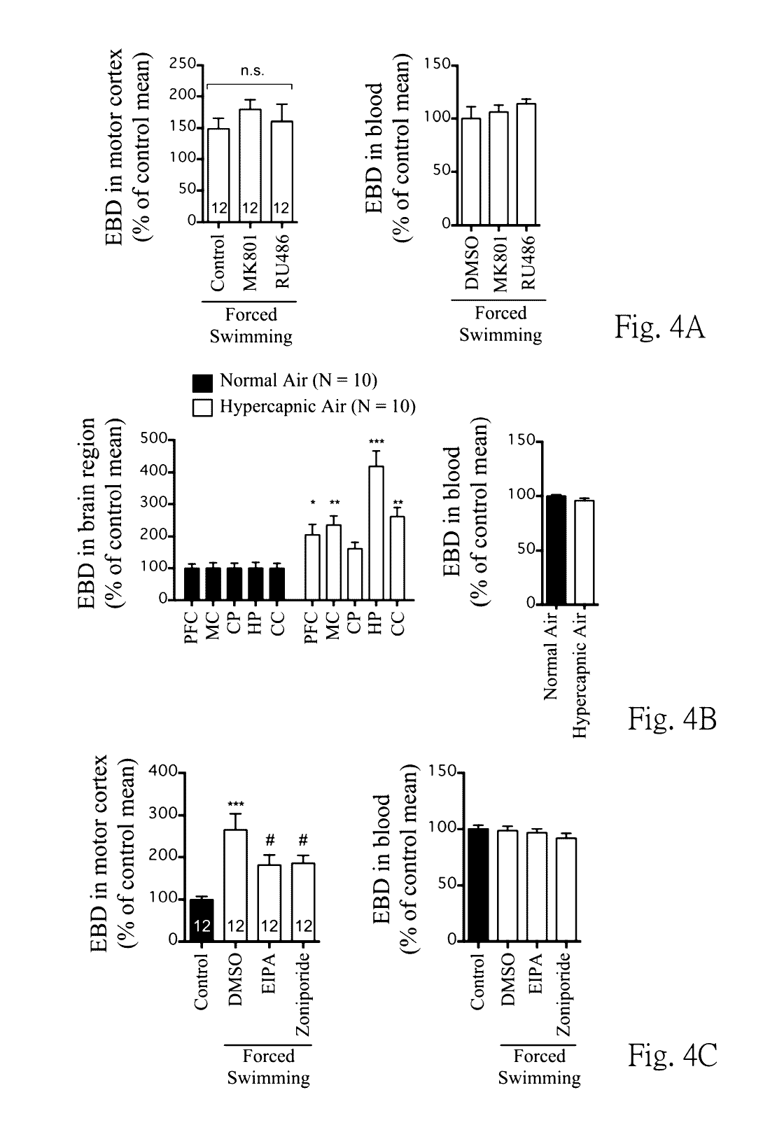

FIG. 4A illustrates the effect of drugs on the blood-brain barrier opening of the rats in the forced swimming test. FIG. 4B is a quantitative diagram showing the detection amount of EBD in different districts of the brain and the blood of the rats in a hypercapnia test. FIG. 4C is a quantitative diagram showing the detection amount of EBD in motor cortex and the blood of the rats injected Na⁺/H⁺ exchanger (NHE).

To test whether the NMDAR pathway contributes to the blood-brain barrier opening, the rats are treated as follow. There are 3 groups in the rats. In the group 1, the rats are treated with DMSO (subcutaneous injection, s.c.) as control group. In the group 2, the rats are treated with a NMDAR blocker MK801 (1 mg/kg, Intraperitoneal injection, i.p., a dose previously shown to inhibit metabolic hyperemia). In the group 3, the rats are treated with RU486 (10 mg/kg, s.c., a dose previously shown to prevent stress-mediated enhancement of NMDAR function). In FIG. 4A, it finds that neither MK801 nor RU486 affect the blood-brain barrier opening during the forced swim test. Thus, neither the glucocorticoid receptor nor the NMDAR contribute appreciably to the forced swimming-related blood-brain barrier opening.

In the brain, the neuronal activity also induces prolonged acidification of an interstitial fluid that can last for minutes, suggesting a role of CO₂/H⁺ in the late phase of functional cerebral hyperemia. However, because this acidification is usually delayed (being preceded by transient alkalization) relative to the onset of hyperemia, acidosis is unlikely to contribute to the early phase of metabolic hyperemia. To examine whether CO₂/H⁺ could trigger the blood-brain barrier opening, the rats injected with EBD are placed inside a chamber filled with either normal air or hypercapnic air 30 minutes, and then quantifying the EBD extravasation into different districts of the brain and the blood of the rats. In FIG. 4B, the rats subjected to hypercapnic acidosis display widespread the EBD extravasation notably, indicating global blood-brain barrier opening. The result of this example raises the intriguing possibility that functional blood-brain barrier opening could be mediated by CO₂/H⁺, which is the same mediator for functional hyperemia in peripheral organs and the late phase of hyperemia in the brain.

Metabolic acidosis can exert its effects via a number of acid-sensitive ion channels or the proton transporters. In particular, the endothelial Na⁺/H⁺ exchanger (NHE) has been implicated in regulating endothelial cell volume and morphology, and thus may be an effector for the blood-brain barrier integrity. To examine whether the NHE contributes to metabolic blood-brain barrier disruption during stressful activity, we administer either a vehicle (i.p.) or an NHE inhibitor (i.p.) to EBD-treated rats prior to forced swimming and quantify the EBD extravasation into the brain. Ethylisopropyl amiloride (EIPA, 5 mg/kg) and zoniporide (20 mg/kg) are selected as NHE inhibitor for this example because they represent two distinct classes of NHE inhibitors and have no known overlapping nonspecific targets. In addition, EIPA and its derivatives have been reported to have little or no effect on Na⁺/H⁺ exchange in forebrain neurons and mammalian astrocytes, yet they strongly affect endothelial Na⁺/H⁺ exchange and NHE-mediated change in endothelial cell volume. In FIG. 4C, inhibition of NHE with either inhibitor attenuates the EBD extravasation into the motor cortex during the forced swimming test.

(4). Stress Associated with Behavior Activity does not Contribute to Acute Blood-Brain Barrier Opening

Stress is known to cause delayed the blood-brain barrier disruption, and this has been proposed to explain central drug side effects observed in Gulf war veterans. Given that the elevated platform test and the forced swim test are previously shown to cause stress in the test animals, we next examine whether stress per se could contribute to opening of the blood-brain barrier in this example.

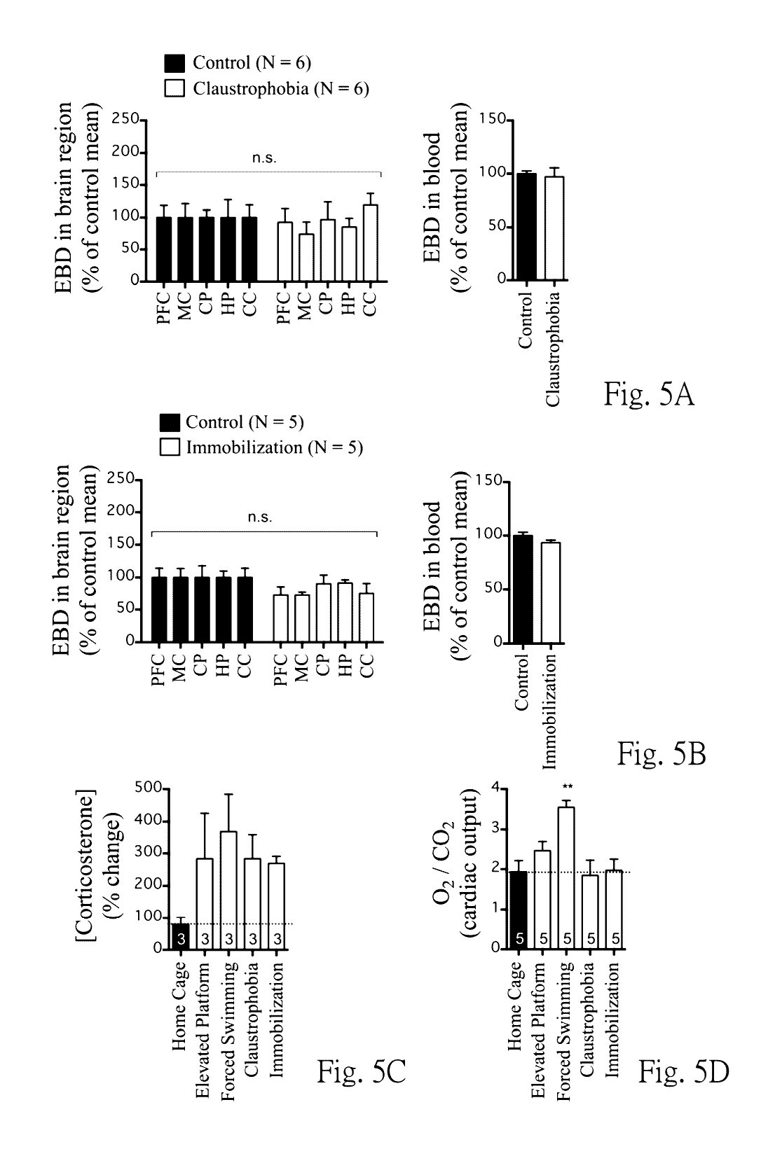

FIGS. 5A-5D show the results of stress associated with behavior activity tests. FIG. 5A is a quantitative diagram showing the detection amount of EBD in different districts of the brain and the blood of the rats in a claustrophobia test. FIG. 5B is a quantitative diagram showing the detection amount of EBD in different districts of the brain and the blood of the rats in an immobilization test. FIG. 5C illustrates concentration changes of corticosterone in the rats in stress associated with behavior activity test. FIG. 5D illustrates O₂/CO₂ content in the rats in stress associated with behavior activity test.

The rats are subjected to claustrophobic stress or restrain stress. Both of these stress tests are unique in that they limited motor activity and prohibited spatial observation; hence the rats are under a claustrophobic stress and a restrain stress. In FIGS. 5A and 5B, the detection amount of EBD in different districts of the brain of the rats has no significant differences between the experiment groups, the claustrophobia test and the immobilization test, and the control groups. Nevertheless, in FIG. 5C, the rats produce rise in corticosterone, a hormonal indicator of stress, equivalent to that produced by the elevated platform test or the forced swim test. Although severe stress caused by prolonged and/or chronic exposure to various stressors, including restrain stress, is known to disrupt the blood-brain barrier in a delayed manner. It does not find the blood-brain barrier opening in the rats subjected to acute claustrophobic stress or restrain stress, Therefore, the data of this example suggests that acute the blood-brain barrier opening caused by the elevated platform test and the forced swim test could not be caused by stress alone.

Prostaglandin and serotonin are previously implicated in stress-mediated blood-brain barrier disruption. To examine whether prostaglandin and serotonin could contribute to opening of the blood-brain barrier, the rats are treated with indomethacin (10 mg/kg, i.p.) and cyproheptadine (15 mg/kg, i.p.) and then performing the forced swimming test, wherein indomethacincan can reduce the synthesis of prostaglandin and cyproheptadine competes with serotonin in that it acts on a postsynaptic receptor. The number of the rats that remain inactive during the forced swimming test, as noted by an observer blinded to the treatment, is 0/6 for DMSO (vehicle), 2/6 for indomethacin, 4/6 for cyproheptadine, and 0/6 for EIPA. The data shows that the doses of indomethacin and cyproheptadine used in this example produce marked sedation in the rats. In comparison, the doses of the vehicle and EIPA (5 mg/kg, i.p.) used in this example are not sedative. Thus, previously reported inhibitory effect of indomethacin and cyproheptadine on the blood-brain barrier disruption is consistent with the neuronal activity dependence reported in this example and is not likely the direct result of prostaglandin or serotonin inhibition.

(5). Aerobic Respiration Potentiates CO₂/H⁺-Mediated Blood-Brain Barrier Opening

Given the sensitivity of the blood-brain barrier to changes in osmotic pressure and the role of NHE in functional blood-brain barrier opening (FIG. 4C). We measure blood Na⁺ levels and an osmolarity of the rats using a blood gas/electrolyte analyzer in this example. The data of this example is shown in Table 1 and Table 2 as follows and FIG. 5D, wherein **, ***, # and ## represents p<0.01, p<0.001, p<0.05 and p<0.01, respectively.

TABLE 1

| | Group | | | | | |
|---|---|---|---|---|---|---|
| | | Elevated | Forced | | | |
| | Control | Platform | Swimming | Claustrophobia | Immobilization | 1-way ANOVA |
| Sample size (N) | N = 5 | N = 5 | N = 5 | N = 5 | N = 5 | |

Gas

| | | | | | | |
|---|---|---|---|---|---|---|
| pH | 7.46 ± 0.01 | 7.47 ± 0.01 | 7.37 ± 0.01*** | 7.44 ± 0.01 | 7.44 ± 0.01 | P < 0.0001 |
| pCO₂ (mmHg) | 37 ± 1 | 32 ± 1 | 44 ± 2* | 37 ± 3 | 36 ± 1 | P = 0.0009 |
| pO₂ (mmHg) | 71 ± 9 | 79 ± 6 | 158 ± 11*** | 65 ± 10 | 71 ± 9 | P < 0.0001 |

Electrolyte

| | | | | | | |
|---|---|---|---|---|---|---|
| Na⁺ (mM) | 136 ± 1 | 135 ± 2 | 137.6 ± 0.1 | 137 ± 1 | 138 ± 1 | P = 0.5254 |
| K⁺ (mM) | 4.9 ± 0.2 | 5.1 ± 0.2 | 4.0 ± 0.1** | 4.9 ± 0.1 | 4.9 ± 0.2 | P = 0.0009 |
| Cl⁻ (mM) | 107 ± 2 | 108 ± 3 | 104 ± 0.5 | 106 ± 2 | 104 ± 0.5 | P = 0.5150 |
| Ca⁺ (mM) | 1.15 ± 0.04 | 1.07 ± 0.06 | 1.10 ± 0.09 | 1.07 ± 0.08 | 1.16 ± 0.07 | P = 0.7958 |
| Mg²⁺ (mM) | 0.36 ± 0.01 | 0.36 ± 0.03 | 0.37 ± 0.03 | 0.37 ± 0.02 | 0.37 ± 0.02 | P = 0.9938 |
| Osm (mOsm/kg) | 275 ± 3 | 274 ± 5 | 283 ± 1 | 277 ± 2 | 280 ± 1 | P = 0.2255 |

TABLE 2

| | Group | | | | | |
|---|---|---|---|---|---|---|
| | | Elevated | Forced | | | |
| | Control | Platform | Swimming | Claustrophobia | Immobilization | 1-way ANOVA |
| Sample size (N) | N = 6 | N = 3 | N = 3 | N = 3 | N = 3 | |

Within 30 minutes post-activity

| | | | | | | |
|---|---|---|---|---|---|---|
| pH | 7.40 ± 0.02 | 7.38 ± 0.01 | 7.35 ± 0.03 | 7.40 ± 0.01 | 7.38 ± 0.02 | P = 0.3709 |
| pCO₂ (mmHg) | 41 ± 3 | 41 ± 2 | 46 ± 9 | 42 ± 2 | 37 ± 1 | P = 0.7772 |
| pO₂ (mmHg) | 85 ± 3 | 93 ± 4 | 131 ± 8*** | 78 ± 2 | 85 ± 2 | P < 0.0001 |

90-120 minutes post-activity

| | | | | | | |
|---|---|---|---|---|---|---|
| pH | 136 ± 1 | 135 ± 2 | 137.6 ± 0.1 | 137 ± 1 | 138 ± 1 | P = 0.2125 |
| pCO₂ (mmHg) | 4.9 ± 0.2 | 5.1 ± 0.2 | 4.0 ± 0.1** | 4.9 ± 0.1 | 4.9 ± 0.2 | P = 0.7401 |
| pO₂ (mmHg) | 107 ± 2 | 108 ± 3 | 104 ± 0.5 | 106 ± 2 | 104 ± 0.5 | P = 0.0014 |

Paired t-test (comparing the 2 time points)

| | | | | | |
|---|---|---|---|---|---|
| pH | P = 0.9185 | P = 0.1781 | P = 0.3981 | P = 0.0861 | P = 0.3825 |
| pCO₂ (mmHg) | P = 0.8507 | ^{##}P = 0.0024 | P = 0.8300 | P = 0.3639 | P = 0.4777 |
| pO₂ (mmHg) | P = 0.4291 | ^{#}P = 0.0476 | P = 0.2775 | P = 0.4600 | P = 0.0627 |

The data of this example finds that blood Na⁺ levels and the osmolarity of the rats no change in either following behavioral activities used in this example. Unexpectedly, the blood gas data revealed increased blood pO₂ in the rats subjected to the elevated platform (P>0.05) and the forced swim (P<0.0001) tests (Table 1 and FIG. 5D), and this increase appeared to decay over time (P=0.0476 for the elevated platform test; P=0.2775 for the forced swimming test) (Table 2). Therefore, we explored the possibility that increased pO₂ might directly affect the blood-brain barrier permeability in this example.

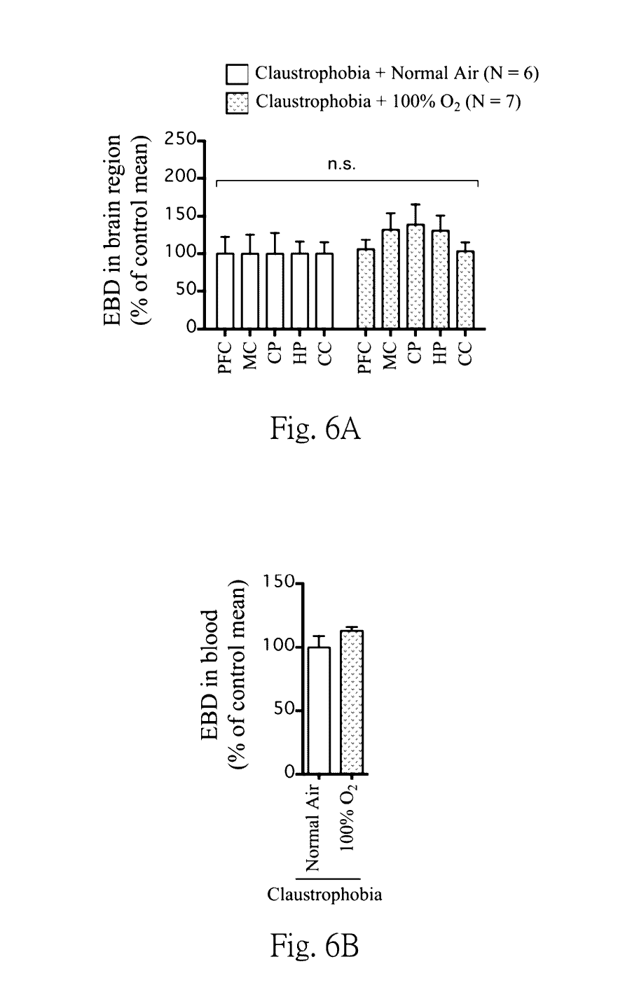

FIGS. 6A and 6B show the effects of increased pO₂ on the blood-brain barrier permeability. FIG. 6A is a quantitative diagram showing the detection amount of EBD in different districts of the brain of the rats in the claustrophobia test. FIG. 6B is a quantitative diagram showing the detection amount of EBD in the blood of the rats in the claustrophobia test. The rats are placed in gas chambers and allowed to breathe either normal air or 100% O₂, and then performing the claustrophobia test. In FIG. 6A, although there appeared to be some increase in the blood-brain barrier permeability according to the EBD extravasation in the motor cortex, the caudate/putamen, and the hippocampus, the increase is not significant (P>0.05). This finding suggests that increases in pO₂ alone do not account for the observed differences in the blood-brain barrier permeability following behavioral activity.

In FIG. 4B, given that the blood-brain barrier opening can be triggered by CO₂/H⁺, we next determine whether increased pO₂ might potentiate the blood-brain barrier opening by the hypercapnia. The rats are anesthetized with urethane to temporarily halt behavioral activities; after anesthetization, the rats are grouped and received different gas sources 90 minutes, and then quantifying the EBD extravasation into different districts of the brain of the rats. The gas sources are normal air (20% O₂), 100% O₂, air with increased CO₂ (20% O₂, 20% CO₂), and 20% carbogen (20% CO₂ with 80 era O₂).

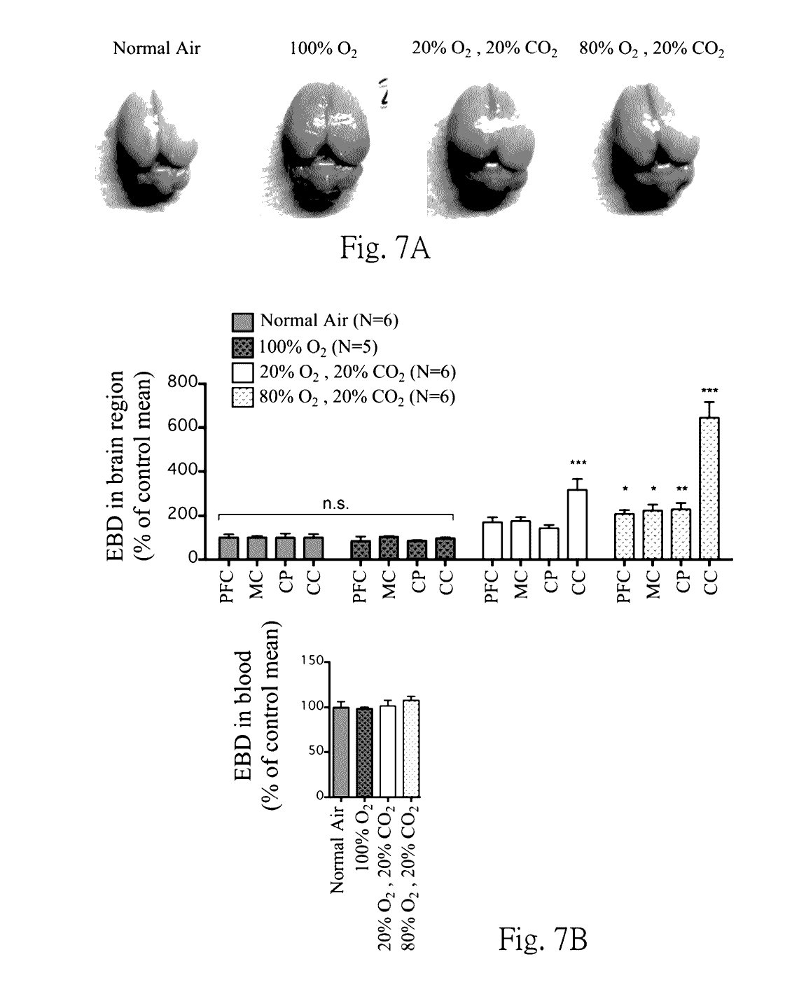

The data of this example is shown in Table 3 as follows and FIGS. 7A-7B. Table 3 shows the effects of different gas sources inhalation on p, pCO₂, and pH value of the blood of the rats confirmed using the blood gas analysis. FIG. 7A-7B show the effects of the hypercapnia O₂-containing gas source inhalation on the blood-brain barrier permeability of the rats. FIG. 7A is a photograph showing the EBD extravasation in the rats breathed different gas sources. FIG. 7B is a quantitative diagram showing the detection amount of EBD in different districts of the brain and the blood of the rats.

TABLE 3

| Gas | | | |
|---|---|---|---|
| | | 20% O₂; | |
| Normal Air | 100% O₂ | 20% CO₂ | 20% carbogen |
| Sample size (N) | | | |
| N = 2 | N = 2 | N = 2 | N = 2 |

45 minutes of gas exposure

| | | | | |
|---|---|---|---|---|
| pH | 7.3 | 7.3 | 7.0 | 7.0 |
| pCO₂ (mmHg) | 55 | 48 | 136 | 134 |
| pO₂ (mmHg) | 83 | 361 | 108 | 384 |

90 minutes of gas exposure

| | | | | |
|---|---|---|---|---|
| pH | 7.3 | 7.4 | 7.0 | 7.0 |
| pCO₂ (mmHg) | 54 | 52 | 133 | 128 |
| pO₂ (mmHg) | 70 | 442 | 96 | 378 |

In Table 3, although pO₂ alone has no effect on the blood-brain barrier permeability, it substantially potentiates pCO₂/H⁺-mediated blood-brain barrier disruption, as shown by marked EBD extravasation in FIGS. 7A and 7B.

A regional metabolic acidosis is caused by a local brain activity, but more global metabolic acidosis can also be induced by a hypoventilation. Given that increased blood pO₂ substantially potentiates the blood-brain barrier opening by CO₂/H⁺, we further examine whether increasing pO2 in the rats subjected to the hypoventilation could open the blood-brain barrier in this example. The rats are injected with EBD first, and the rats are grouped and treated with a respiratory treatment by different levels of ventilation. Next, quantifying the EBD extravasation into different districts of the brain of the rats after the respiratory treatment. The respiratory treatment are a physiologically rate, the hypoventilation and a general ventilation, wherein a respiratory rate of the physiologically rate is 90 BPM (breath per minute) by using a tracheotomy, the respiratory rate of the general ventilation is 80 BPM, and the respiratory rate of the hypoventilation is 50 BPM. The rats of the hypoventilation group are further divided into 2 groups, one group breathing normal air and another group breathing 100% O₂.

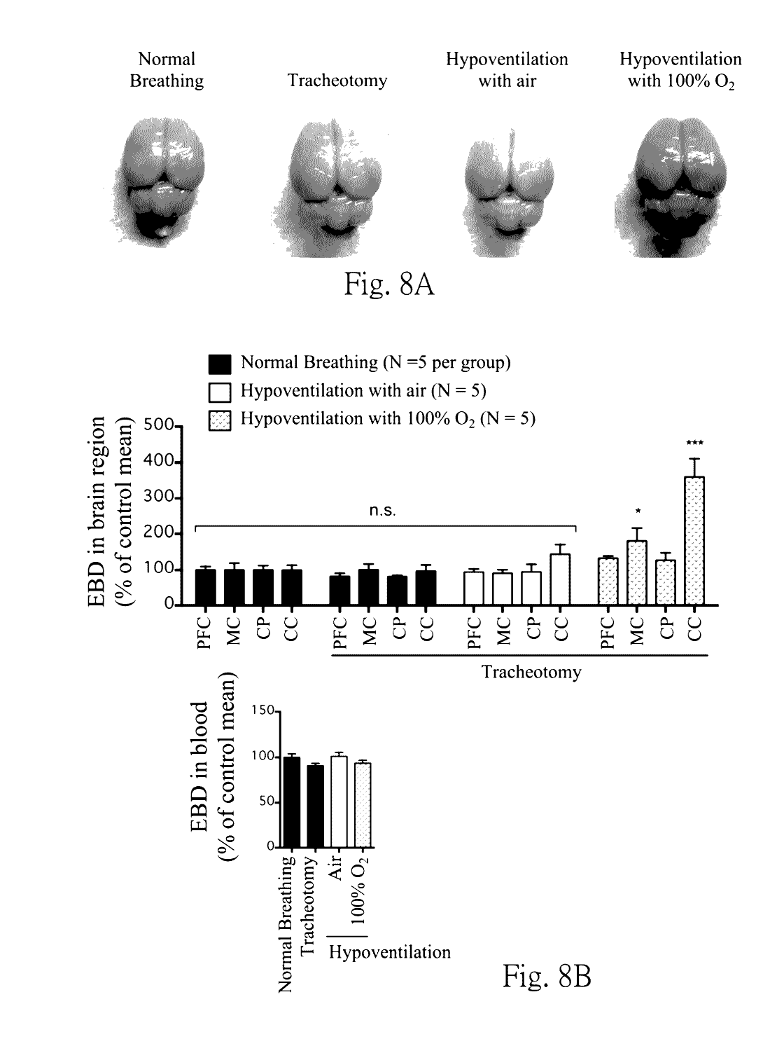

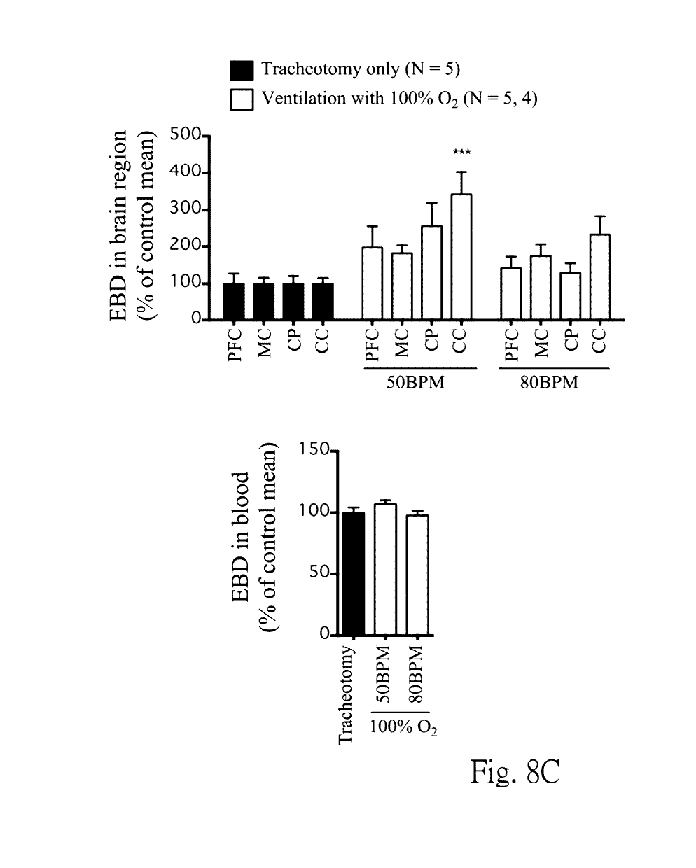

The data of the respiratory treatment is shown in Table 4 as follows and FIG. 8A-8C. Table 4 shows arterial blood gas and electrolyte content of the rats following different levels of ventilation. FIGS. 8A-8C show the effects of different levels of ventilation on the blood-brain barrier permeability. FIG. 8A is a photograph showing the EBD extravasation in the rats following different levels of ventilation. FIG. 8B is a quantitative diagram showing the detection amount of EBD in different districts of the brain and the blood of the rats in the hypoventilation. FIG. 8C is a quantitative diagram showing the detection amount of EBD in different districts of the brain and the blood of the rats increased a ventilation rate.

TABLE 4

| Group | | |
|---|---|---|
| Spontaneous | Hypoventilation | Hypoventilation with |
| Breathing | with normal air | 100% O₂ |
| Sample size (N) | | |
| N = 2 | N = 2 | N = 2 |

45 minutes of gas exposure

| | | | |
|---|---|---|---|
| pH | 7.4 | 7.0 | 7.0 |
| pCO₂ (mmHg) | 43 | 89 | 118 |
| pO₂ (mmHg) | 80 | 52 | 389 |

90 minutes of gas exposure

| | | | |
|---|---|---|---|
| pH | 7.4 | 7.0 | 7.0 |
| pCO₂ (mmHg) | 48 | 98 | 130 |
| pO₂ (mmHg) | 76 | 52 | 389 |

In Table 4, FIGS. 8A and 8B, the hypoventilation with 100% O₂ increases both pO₂ and pCO₂ and opens the blood-brain barrier, whereas the hypoventilation with normal air increases only pCO₂ and has no effect on the blood-brain barrier permeability compared with that of normal breathing. Likewise in FIG. 8C, increasing the ventilation rate attenuates the blood-brain barrier opening by O₂ inhalation, confirming that systemic accumulation of CO₂/H⁺ during the hypoventilation is required for the blood-brain barrier opening. The data of the respiratory treatment demonstrate that increased blood PO₂ during aerobic activity can potentiate metabolic acidosis-induced the blood-brain barrier opening and explain why non-aerobic activity only resulted in minor the blood-brain barrier opening.

### II. Gas Source Increases the Blood-Brain Barrier Permeability

The data of the first part examples demonstrate that increased blood pO₂ during aerobic activity can induce the blood-brain barrier opening. Therefore this part of the examples further discuss that how the gas source regulates the blood-brain barrier opening, whether the blood-brain barrier is reversible, whether the inhalation of the gas source causes adverse effects on the individual, and whether the inhalation of the gas source can help to deliver biological agents to the brain.

### (1). Gas Source Inhalation Disrupts the Blood-Brain Barrier in a Partial Pressure and Exposure Time Dependent Manner

To examine the partial pressure of inhaled gas source necessary to effectively disrupt the blood-brain barrier, the rats are placed inside the gas chamber filled with the gas source, wherein the gas source is normal air, 5% carbogen (5% CO₂, 95% O₂), 10% carbogen (10% CO₂, 90% O₂), or 20% carbogen (20% CO₂, 80% O₂). To quantify the blood-brain barrier permeability, the rats are intravenously injected with EBD, and the amount of EBD extravasated into a brain parenchyma at 90 minutes following the gas source inhalation is quantified. The 90 minutes inhalation time is selected to allow for sufficient EBD extravasation for visual and spectroscopic quantification.

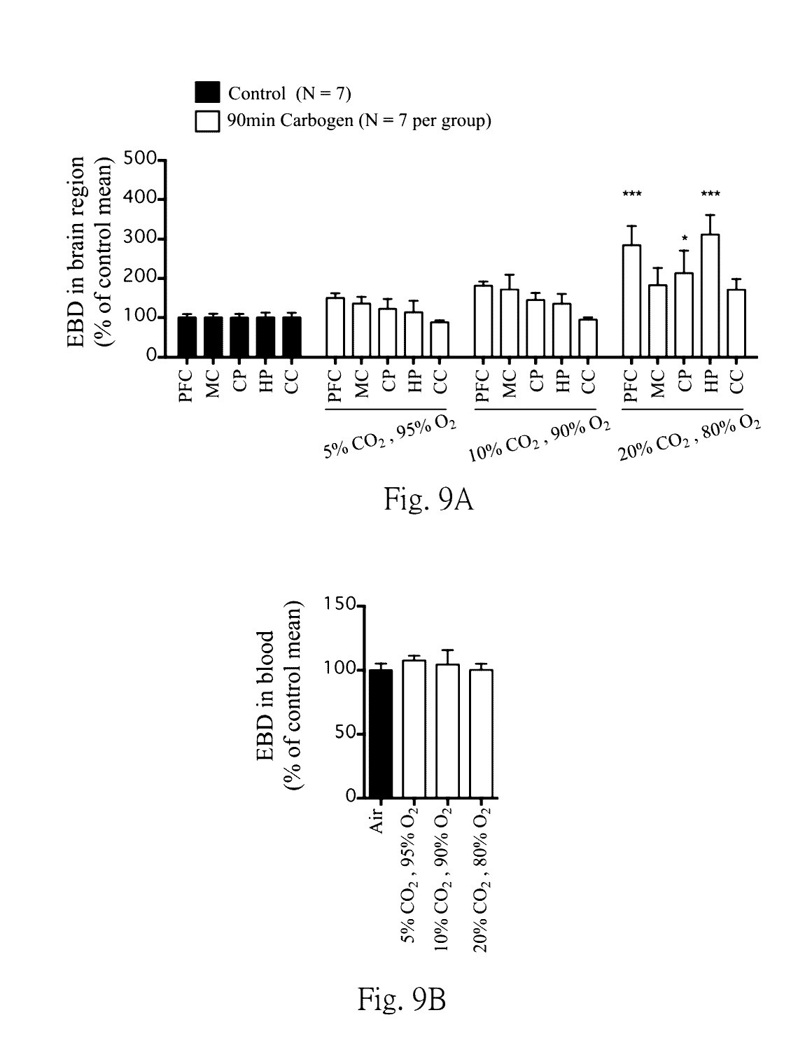

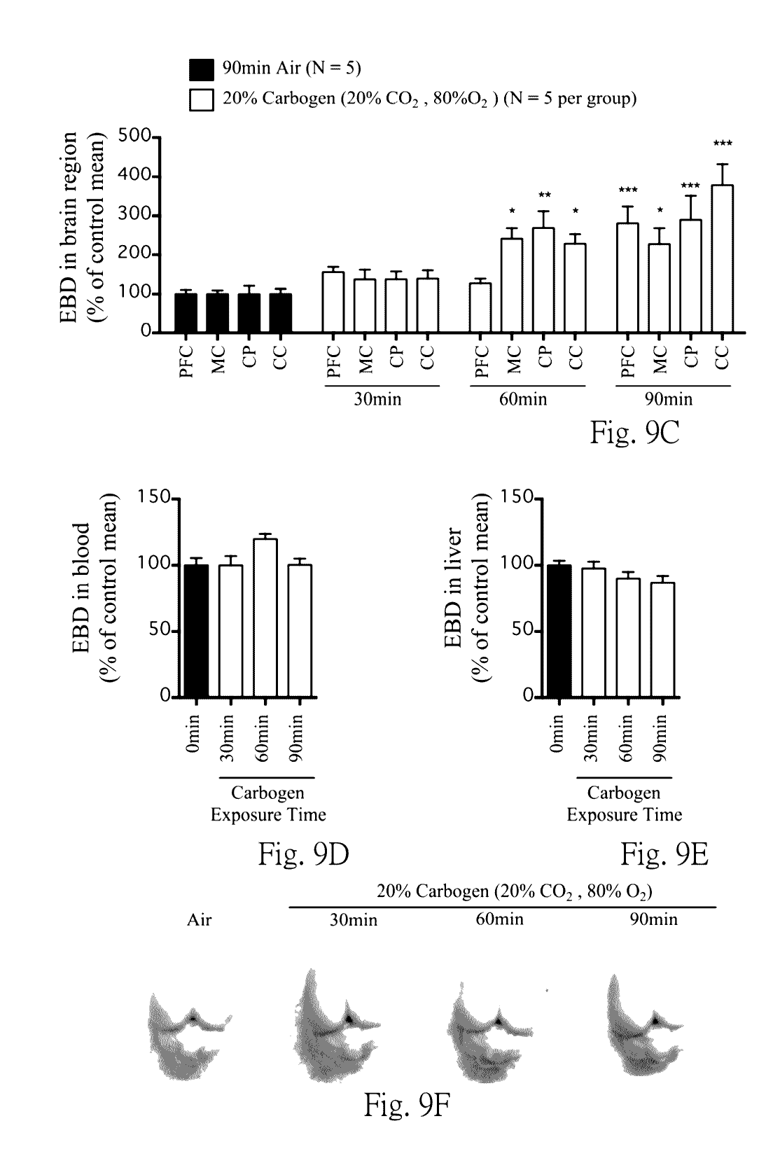

FIGS. 9A-9F show the effects of the inhalation of the gas source on the blood-brain barrier opening of the rats. FIG. 9A is a quantitative diagram showing the detection amount of EBD in different districts of the brain of the rats inhaled different gas sources. FIG. 9B is a quantitative diagram showing the detection amount of EBD in the blood of the rats inhaled different gas sources. FIG. 9C is a quantitative diagram showing the detection amount of EBD in different districts of the brain of the rats inhaled 20% carbogen for different exposure time. FIG. 9D is a quantitative diagram showing the detection amount of EBD in the blood of the rats inhaled 20% carbogen for different exposure time. FIG. 9F is a photograph showing the EBD extravasation in the rats inhaled 20% carbogen for different exposure time.

In FIG. 9A, the rats with slight the blood-brain barrier disruption occurring even following inhalation of only 5% carbogen. The most pronounced blood-brain harrier disruption occurred in response to 20% carbogen, which markedly increased EBD extravasation into the brain and had no effect on EBD concentration in the blood. The result indicates that the carbogen increased the blood-brain barrier permeability to EBD in a partial pressure-dependent manner (P<0.0001).

To further investigate the speed at which the blood-brain barrier is disrupted by a carbogen inhalation, we exposed the rats to the gas chamber filled with 20% carbogen for 30, 60, and 90 minutes. The rats receive EBD injections prior to 20% carbogen exposure and are returned to their home cage immediately following exposure, allowing a total period of 90 minutes for the EBD extravasation. In FIGS. 9C and 9F, the rats with 30 minutes of 20% carbogen exposure leading to minor EBD staining in the brain and 90 minutes of 20% carbogen exposure leading to strong EBD staining in the brain. The results of this example is consistent with the time-dependent extravasation of the EBD, a carbogen-mediated blood-brain barrier disruption is exposure time-dependent (P<0.0001). In FIGS. 9D and 9E, in contrast with its effects on the blood-brain barrier permeability, the carbogen had no effect on the level of EBD in the blood or on its extravasation into the liver.

### (2). Gas Source-Induced Blood-Brain Barrier Disruption is Rapidly Reversible

One major problem associated with therapeutic blood-brain barrier disruption is poor reversibility in current methods. The blood-brain barrier disruption via an intracarotid injection of hyperosmolar solutions requires over 5 hours of recovery time in the rats and over 6 h in humans. Intravenous treatments to disrupt the blood-brain barrier have shown variable persistence that may be difficult to control, which could explain discrepancies between laboratory findings and clinical results in the central delivery of chemotherapeutic drugs.

To examine whether the gas source-mediated blood-brain barrier disruption is reversible, the rats are divided into 9 groups. The rats first are divided into 3 groups to inhale 20% carbogen for 30, 60, or 90 minutes, respectively. Each group is further divided into 3 groups, one group injected with EBD immediately, another group injected with EBD 1 hour following the inhalation of 20% carbogen, and the other group injected with EBD 24 hours following the inhalation of 20% carbogen, and then quantified the EBD extravasation 90 minutes after the injection. The control group is injected with EBD following the inhalation of normal air, and then quantified the EBD extravasation 90 minutes after the injection.

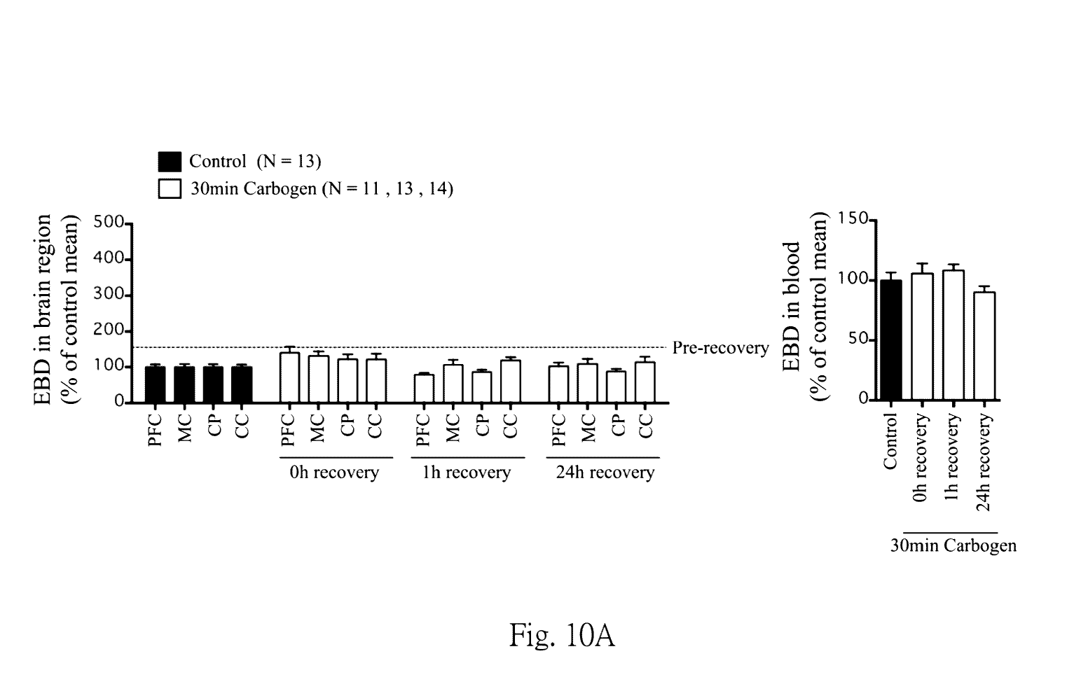

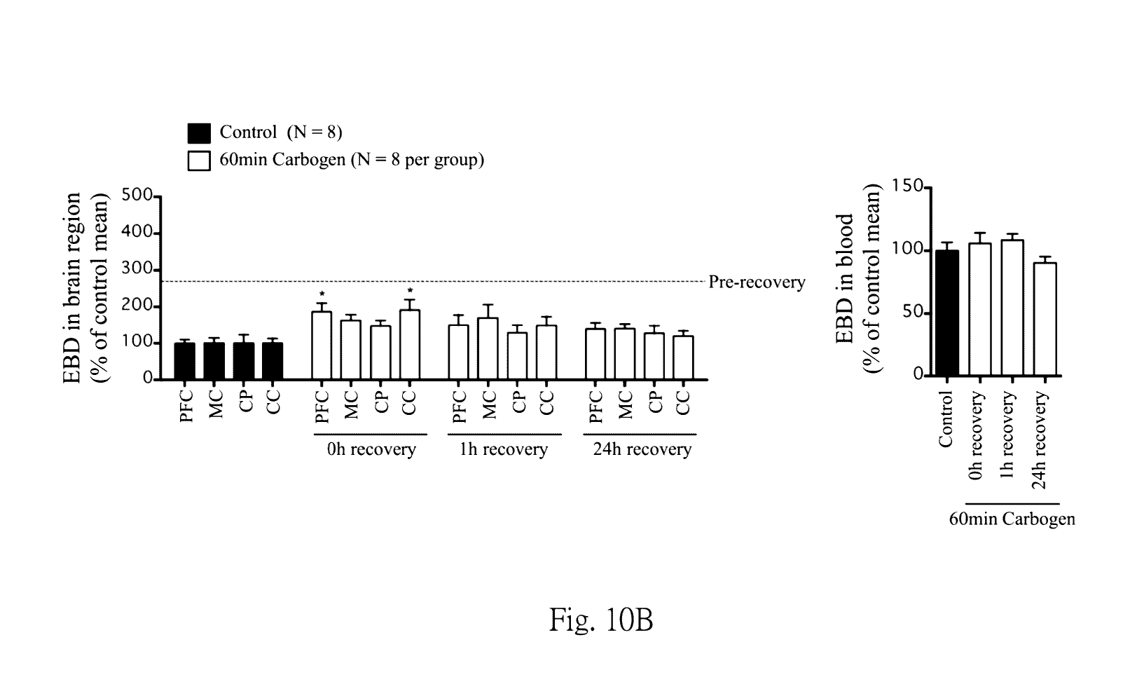

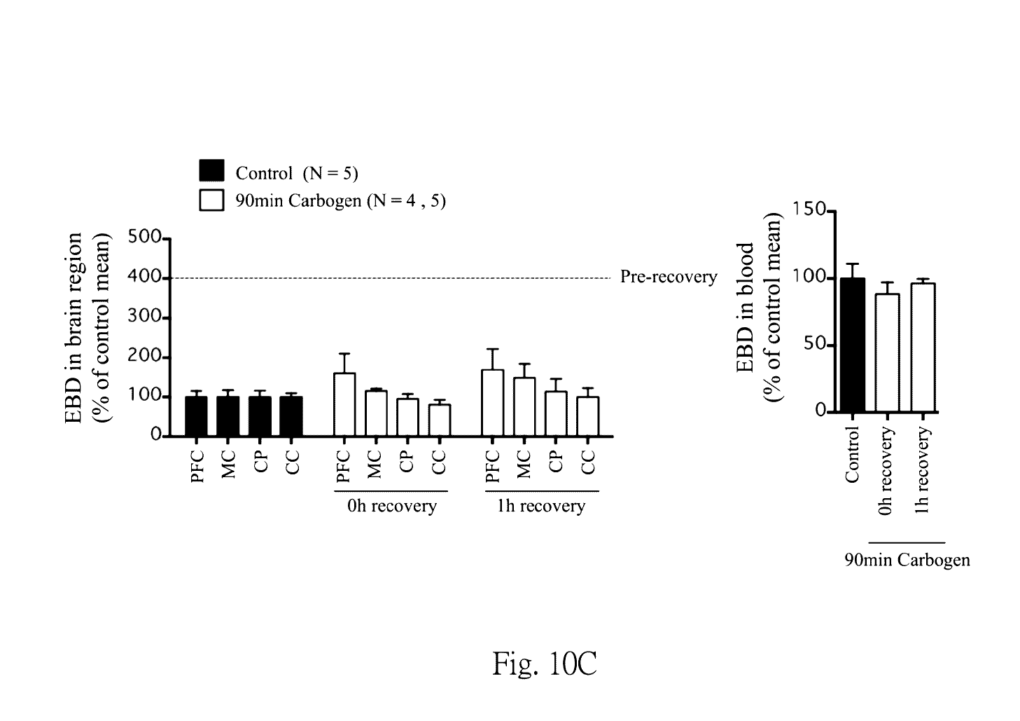

FIGS. 10A-10C show the results of a reversibility test on regulation of the blood-brain barrier opening. FIG. 10A is a quantitative diagram showing the detection amount of EBD in different districts of the brain and the blood of the rats during recovery from 30 minutes of 20% carbogen inhalation. FIG. 10B is a quantitative diagram showing the detection amount of EBD in different districts of the brain and the blood of the rats during recovery from 60 minutes of 20% carbogen inhalation. FIG. 10C is a quantitative diagram showing the detection amount of EBD in different districts of the brain and the blood of the rats during recovery from 90 minutes of 20% carbogen inhalation. The results indicate that the carbogen-mediated blood-brain barrier disruption is rapidly reversible, with evidence of recovery from the carbogen-mediated blood-brain barrier disruption observed immediately after the rats are removed from a carbogen-filled chamber. In FIG. 10A, following 30 minutes of the carbogen exposure, the blood-brain barrier permeability recovered from 150% to 100% of the baseline level by 1 hour. In FIG. 10B following 60 minutes of the carbogen exposure, the blood-brain barrier permeability recovered from 250% to 100% of the baseline level by 24 hours. In FIG. 10C, following 90 min of the carbogen exposure, the blood-brain barrier permeability to the EBD extravasation recovered from 400% to 100-150% of the baseline level.

(3). Gas Source-Mediated Blood-Brain Barrier Disruption does not Cause Metabolic and Cognitive Deficit

Safety concerns are of utmost importance during clinical application. Experimental evidence has shown that transient blood-brain barrier disruption alone does not cause neurological injury, but poorly performed procedures to disrupt the blood-brain barrier can be injurious. For example, the blood-brain barrier disruption by the intracarotid injection of the hyperosmolar solution is already a common protocol used to deliver chemotherapeutic agents. However, an introduction of these agents requires a technically demanding invasive surgery, in which clinical errors can cause brain injuries. Although the blood-brain barrier disruption via ultrasound does not require invasive surgery, the improper use of ultrasound can cause a capillary damage and a tissue hemorrhage. In comparison to existing methods, the gas source-mediated blood-brain barrier disruption may be a safer alternative. Although the administration of the carbogen to treat central disorders has now largely been replaced by other more effective treatments, its clinical adverse effects following acute and repeated exposure have been well documented.

To examine whether the inhalation of the gas source causes adverse effects on the rats, the rats are grouped and exposed at 20% carbogen for 30, 60, or 90 minutes (Day 0), respectively. We measured body weights of the rats on Days −4, 0, 7, and 18, weekly food intake on Days 0, 7, 14, and 20, a locomotor activity on Day 20, and learning and memory performance (the novel subject recognition test) on Day 22 following the carbogen exposure. Because of technical limitations related to tracer quantitation, the rats are exposed to at least 30-90 minutes of 20% carbogen to obtain quantifiable blood-brain barrier disruption. However, we cannot exclude the possibility that shorter exposure times, such as those that have been administered previously in psychiatric clinics, may not result in the blood-brain barrier disruption.

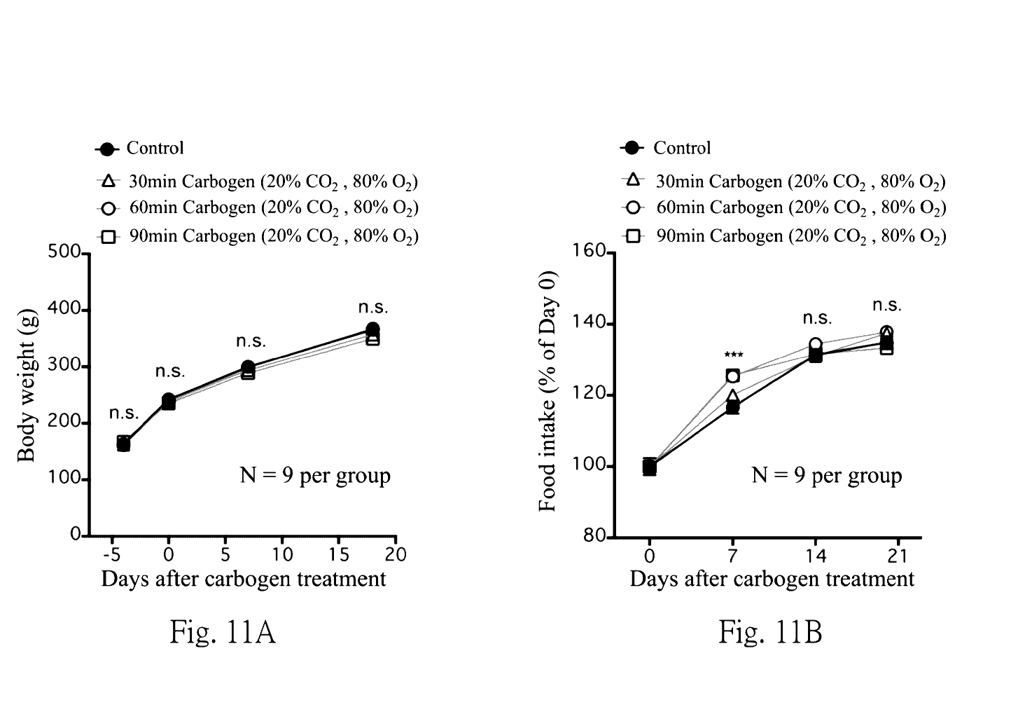

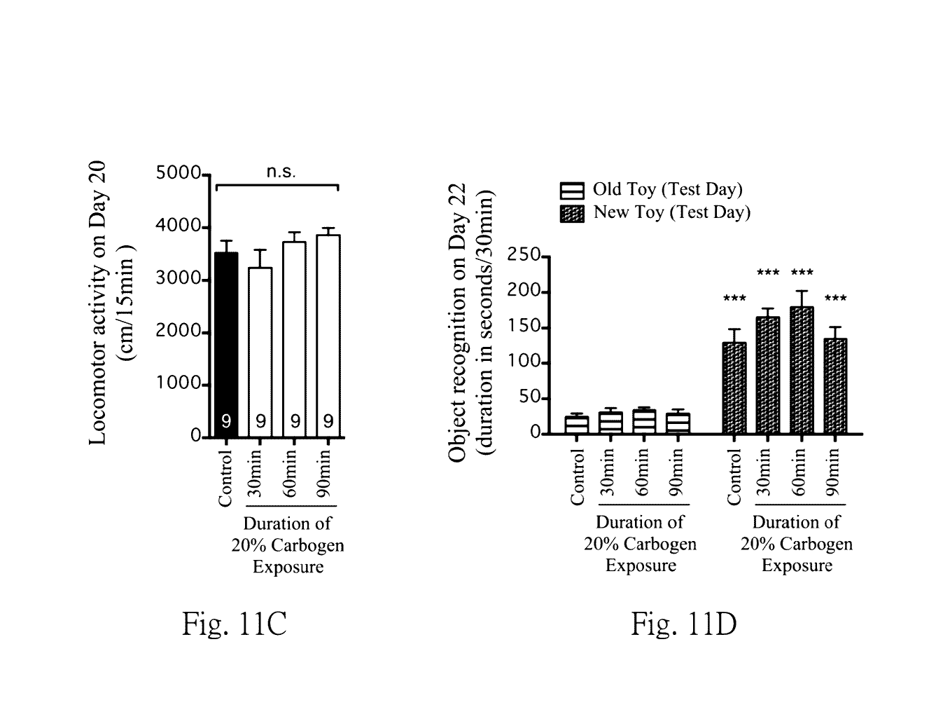

FIGS. 11A-11D show the effects of the inhalation of the gas source on the metabolism and the cognitive function of the rats. FIG. 11A is a quantitative diagram showing body weight changes of the rats inhaled 20% carbogen. FIG. 11B is a quantitative diagram showing the food intake of the rats inhaled 20% carbogen. FIG. 11C is a quantitative diagram showing the result of a locomotor activity on Day 20, wherein the rats inhale 20% carbogen. FIG. 110 is a quantitative diagram of the result of the novel object recognition test on Day 22, wherein the rats inhale 20% carbogen.

In FIGS. 11A and 11B, 30 minutes of the carbogen inhalation has no effect on body weight or food intake compared with those of the control rats. In comparison, 60 and 90 minutes of the carbogen inhalation led to increased food intake during the first week, but the animals returned to their normal behavior patterns by the second week. In addition in FIG. 11C, 30, 60, and 90 minutes of the carbogen exposure have no effect on the locomotor activity, which is a measure of anxiety. In FIG. 11D, 30, 60, and 90 minutes of the carbogen exposure have no effect on learning and memory performance according to the novel object recognition test.

### (4). Gas Source-Induced Blood-Brain Barrier Disruption Assists in Central Delivery of a Memory Erasing Peptide

To assess the effectiveness of the inhalation of the gas source in delivering biological agents to the brain, the rats are injected with the memory-erasing peptide ZIP and exposed to 60 minutes of 20% carbogen, followed by an assessment of memory recall using the object recognition test. The steps of the novel object recognition test are the same as that shown in the example I-2-i, and thus it is not illustrated any further. The rats are randomly divided into 3 groups. On Day 1 of the object recognition test, the rats of group 1 are received the carbogen with a saline, the rats are received a ZIP peptide injection alone, and the rats are received the carbogen with the ZIP peptide injection.

FIGS. 12A-12D show the results of the carbogen-mediated blood-brain barrier disruption helping to deliver the ZIP peptide to the brain. FIG. 12A is a flow schematic view of the novel subject recognition test, wherein the rats received the carbogen inhalation and the ZIP peptide injection on Day1. FIG. 12B is a quantitative diagram of the result of the novel object recognition test on Day 1, FIG. 12C is a quantitative diagram of the result of the novel object recognition test on Day 2, FIG. 12D is a schematic diagram of the result of the novel object recognition test.

These 3 groups show no differences in a toy play (recognition) behavior on the training day (Day 0) (data not shown). But in FIGS. 126 and 12D, the rats of group1 (treated with the carbogen and the saline) and group 2 (treated with the ZIP peptide injection alone) show a greater preference for the new toy over the old toy on the first test day (Day 1). In comparison, the rats of group 3 (treated with the carbogen and the ZIP peptide injection) show no preference for either the new toy or the old toy. We expected both the new toy and the old toy to become “old toys” by the end of the first test day. Thus, to evaluate whether the carbogen and/or the peptide prevented the rats from learning new memories, as shown in FIG. 12A, we expose each rat to a second new toy on the second test day (Day 2). In FIGS. 12C and 12C, the rats exhibited clear recognition of the second new toy regardless of the treatment of ZIP peptide received. The result of this example is consistent with previous findings, in which intrahippocampal injection of ZIP peptide has been shown to erase older memories but not prevent the formation of new memories.

To sum up, the present disclosure provides the use of the gas source which containing oxygen; the gas source can increase the blood-brain barrier permeability. The gas source-mediated blood-brain barrier opening is clinical safety and does not cause metabolic and cognitive deficit of the individual. In addition, the inhalation of the gas source does not require expensive equipment and is easy to operate. Moreover, the inhalation of the gas source can help to deliver biological agents to the brain. Because the gas source-mediated blood-brain barrier opening is reversible, the blood-brain barrier can recover the impermeability rapidly after the delivery of the biological agents into the brain to protect the environment of the brain interfered from foreign substances. Therefore, the gas source of the present disclosure can be used to prepare pharmaceutical compositions to increase the blood-brain barrier permeability.

Although the present disclosure has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present disclosure without departing from the scope or spirit of the disclosure. In view of the foregoing, it is intended that the present disclosure cover modifications and variations of this disclosure provided they fall within the scope of the following claims.

What is claimed is:

1. A method of increasing blood-brain barrier permeability of a subject to a molecule, the method comprising causing the subject to inhale a gas source that is effective for increasing blood-brain barrier permeability to the molecule, wherein the gas source comprises at least 20 weight percent oxygen.

2. The method of claim 1, wherein the gas source further comprises at least 1 weight percent carbon dioxide.

3. The method of claim 2, wherein a ratio of a weight/volume percent of the oxygen to the weight/volume percent of the carbon dioxide ranges from 1:1 to 1:20.

4. The method of claim 2, wherein the gas source comprises 20 weight percent oxygen and 1 weight percent carbon dioxide.

5. The method of claim 2, wherein the gas source comprises 20 weight percent oxygen and 20 weight percent carbon dioxide.

6. The method of claim 2, wherein the gas source consists of 95 weight percent oxygen and 5 weight percent carbon dioxide.

7. The method of claim 2, wherein the gas source consists of 90 weight percent oxygen and 10 weight percent carbon dioxide.

8. The method of claim 2, wherein the gas source consists of 80 weight percent oxygen and 20 weight percent carbon dioxide.

9. The method of claim 1, wherein the gas source is 100 weight percent oxygen.

10. The method of claim 1, wherein the molecule is a diagnostic agent.

11. The method of claim 1, wherein the molecule is a neuropharmaceutical agent.
